(19) United States
(12) Patent Application Publication
(10) Pub. No.: US 2025/0345598 A1
(43) Pub. Date: Nov. 13, 2025

(54) GUIDEWIRE FOR PACING DURING REPLACEMENT HEART VALVE DELIVERY

(71) Applicant: BOSTON SCIENTIFIC SCIMED, INC., MAPLE GROVE, MN (US)

(72) Inventors: Gavin O'Brien, Dublin (IE); Tim O'Connor, Galway (IE); John Lardner, Galway (IE); Chris Cullen, Galway (IE)

(73) Assignee: BOSTON SCIENTIFIC SCIMED, INC., MAPLE GROVE, MN (US)

(21) Appl. No.: 19/203,751

(22) Filed: May 9, 2025

Related U.S. Application Data
(60) Provisional application No. 63/645,286, filed on May 10, 2024.

(51) Int. Cl. A61N 1/05; A61F 2/24

(52) U.S. Cl. CPC A61N 1/0565; A61F 2/2427

(57) ABSTRACT

A guidewire may be adapted for delivering a replacement heart valve implant as well as providing pacing to the heart as part of a process of implanting the replacement heart valve implant. The guidewire includes an elongate shaft including a distal section and a proximal section extending proximally from the distal section. The distal section forms a spiral portion that when unconstrained, includes a proximal spiral portion that curves in a direction and defines a plane and a distal spiral portion that extends out of the plane defined by the proximal spiral. One or more electrodes are disposed within the distal section for pacing.

### CROSS-REFERENCE TO RELATED APPLICATION

This application is a continuation of U.S. Patent Application Ser. No. 63/645,286, filed May 10, 2024, entitled “GUIDEWIRE FOR PACING DURING REPLACEMENT HEART VALVE DELIVERY”, which is incorporated by reference herein in its entirety.

### TECHNICAL FIELD

The present disclosure pertains to medical devices and methods for using medical devices. More particularly, the present disclosure pertains to a guidewire for delivering and implanting a replacement heart valve implant with concurrent pacing.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a guidewire for delivering a replacement heart valve implant. The guidewire includes an elongate shaft including a distal section and a proximal section extending proximally from the distal section. The distal section forms a spiral portion when unconstrained and includes a proximal spiral portion that curves in a direction and defines a plane and a distal spiral portion that extends out of the plane defined by the proximal spiral. An electrode is disposed within the distal section.

Alternatively or additionally, the electrode may be adapted for pacing the heart.

Alternatively or additionally, the electrode may include a first electrode that is disposed within the distal spiral portion.

Alternatively or additionally, the guidewire may further include a second electrode that is disposed within the proximal spiral portion and is electrically isolated from the first electrode.

Alternatively or additionally, one of the first electrode and the second electrode may include an anode electrode and the other of the first electrode and the second electrode may include a cathode electrode.

Alternatively or additionally, the elongate shaft may have an outer diameter that is in a range of about 0.76 millimeters (about 0.030 inches) to about 1.02 millimeters (about 0.040 inches).

Alternatively or additionally, the elongate shaft may have an outer diameter that is about 0.89 millimeters (about 0.035 inches).

Alternatively or additionally, the elongate shaft may include an uninsulated core wire and a polymer-coated coil wrapped around the uninsulated core wire.

Alternatively or additionally, one of the uninsulated core wire and the polymer-coated coil may be electrically connected with the electrode.

Another example may be found in a guidewire for delivering a replacement heart valve implant. The guidewire includes an elongate shaft that has an outer diameter ranging from about 0.76 millimeters (about 0.030 inches) to about 1.02 millimeters (about 0.040 inches) and that includes a distal section adapted for pacing. A distal electrode is disposed within the distal section. A proximal electrode is disposed within the distal section and is spaced apart and electrically isolated from the first electrode. The distal section has an unconstrained profile in which the distal section forms a three dimensional shape adapted to place the distal electrode in contact with the ventricular septum and place the proximal electrode in contact with the left ventricle spaced from the ventricular septum.

Alternatively or additionally, in the unconstrained profile, the distal section may include a proximal spiral portion that defines a plane and a distal spiral portion that curves out of the plane defined by the proximal spiral portion.

Alternatively or additionally, the distal electrode may be disposed within the distal spiral portion and the proximal electrode may be disposed within the proximal spiral portion.

Alternatively or additionally, the elongate shaft may include an uninsulated core wire electrically coupled with one of the distal electrode and the proximal electrode and a polymer-coated coil wrapped around the uninsulated core wire and electrically coupled with the other of the distal electrode and the proximal electrode.

Alternatively or additionally, the proximal spiral portion may have a width that ranges from about 29 millimeters (about 1.14 inches) to about 50 millimeters (about 1.97 inches).

Alternatively or additionally, the distal spiral portion may include a distal tip that extends out of the plane defined by the proximal spiral portion a distance ranging from about 5 millimeters (about 0.197 inches) to about 50 millimeters (about 1.97 inches).

Another example may be found in a guidewire for delivering a replacement heart valve implant. The guidewire includes an elongate shaft including a distal section and a proximal section extending proximally from the distal section. The elongate shaft has an outer diameter ranging from about 0.76 millimeters (about 0.030 inches) to about 1.02 millimeters (about 0.040 inches). The distal section forms a spiral portion when unconstrained, the spiral portion including a proximal spiral portion that curves in a direction and defines a plane and a distal spiral portion that extends out of the plane defined by the proximal spiral. A proximal electrode is disposed within the proximal spiral portion and a distal electrode is disposed within the distal spiral portion.

Alternatively or additionally, the proximal electrode and the distal electrode together may be adapted for bipolar pacing of the heart.

Alternatively or additionally, the elongate shaft includes an uninsulated core wire electrically coupled with one of the distal electrode and the proximal electrode and a polymer-coated coil wrapped around the uninsulated core wire and electrically coupled with the other of the distal electrode and the proximal electrode.

Alternatively or additionally, the proximal spiral portion may have a width that ranges from about 29 millimeters (about 1.14 inches) to about 50 millimeters (about 1.97 inches).

Alternatively or additionally, the distal spiral portion may include a distal tip that extends out of the plane defined by the proximal spiral portion a distance ranging from about 5 millimeters (about 0.197 inches) to about 50 millimeters (about 1.97 inches).

The above summary of some embodiments, aspects, and/or examples is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative guidewire disposed within a patient's heart;

FIG. 2A is a perspective view of the illustrative guidewire of FIG. 1;

FIG. 2B is a side view of the illustrative guidewire of FIG. 1;

FIG. 2C is a top plan view of an illustrative guidewire;

FIG. 3A is a perspective view of an illustrative guidewire;

FIG. 3B is a side view of the illustrative guidewire of FIG. 3A;

FIG. 3C is an enlarged view of a portion of the elongate shaft forming part of the illustrative guidewire of FIG. 3A;

FIGS. 4 through 9 are schematic views showing a method of pacing while delivering a replacement heart valve implant using the illustrative guidewire of FIG. 1.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications may be disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of a device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

The terms “transaortic valve implantation” and “transcatheter aortic valve implantation” may be used interchangeably and may each be referred to using the acronym “TAVI”. The terms “transaortic valve replacement” and “transcatheter aortic valve replacement” may be used interchangeably and may each be referred to using the acronym “TAVR”. The terms TAVI and TAVR may be used to refer to the same or similar procedures and in at least some embodiments, the terms TAVI and TAVR may be used interchangeably.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. Treatment of defective heart valves poses other challenges in that the treatment often requires the repair or outright replacement of the defective valve. Such therapies may be highly invasive to the patient. Disclosed herein are medical devices and/or procedures that may be used within a portion of the cardiovascular system in order to diagnose, treat, and/or repair the system, for example during and/or in conjunction with a TAVI or TAVR procedure, or in place of a TAVI or TAVR procedure in patients not suitable for such. At least some of the medical devices and/or procedures disclosed herein may be delivered and/or performed percutaneously and, thus, may be much less invasive to the patient, although other surgical methods and approaches may also be used. The devices disclosed herein may also provide a number of additional desirable features and benefits as described in more detail below. For the purpose of this disclosure, the discussion below is directed toward the treatment of a native aortic valve and will be so described in the interest of brevity. This, however, is not intended to be limiting as the skilled person will recognize that the following discussion may also apply to a mitral valve or another heart valve with no or minimal changes to the structure and/or scope of the disclosure. Similarly, the medical devices and/or procedures disclosed herein may have applications and uses in other portions of a patient's anatomy, such as but not limited to, arteries, veins, and/or other body lumens.

A guidewire may be adapted for delivering a replacement heart valve implant while also being adapted to permit pacing the heart using the guidewire. The guidewire may include an elongate shaft including a distal section and a proximal section that extends proximally from the distal section. The distal section forms a spiral portion when unconstrained. The coiled portion includes a proximal spiral portion that curves in a direction and defines a plane and a distal spiral portion that extends out of the plane defined by the proximal spiral. An electrode is disposed within the distal section. In some cases, the electrode may be adapted for pacing the heart. In some cases, the electrode may be a first electrode and may be disposed within the distal spiral portion. The guidewire may further include a second electrode that is disposed within the proximal spiral portion and is electrically isolated from the first electrode. One of the first electrode and the second electrode may include an anode electrode and the other of the first electrode and the second electrode may include a cathode electrode.

In some cases, the elongate shaft may have an outer diameter that is in a range of about 0.76 millimeters (about 0.030 inches) to about 1.02 millimeters (about 0.040 inches). As an example, the elongate shaft may have an outer diameter that is about 0.89 millimeters (about 0.035 inches). In some cases, the elongate shaft may include an uninsulated core wire and a polymer-coated coil that is wrapped around the uninsulated core wire. In some cases, one of the uninsulated core wire and the polymer-coated coil may be electrically connected with the electrode.

A guidewire may be adapted for delivering a replacement heart valve implant while also being adapted to permit pacing the heart using the guidewire. The guidewire may include an elongate shaft having an outer diameter ranging from about 0.76 millimeters (about 0.030 inches) to about 1.02 millimeters (about 0.040 inches). The elongate shaft may include a distal section adapted for pacing. A distal electrode is disposed within the distal section and a proximal electrode is disposed within the distal section. The proximal electrode is spaced apart and electrically isolated from the first electrode. The distal section has an unconstrained profile in which the distal section forms a three dimensional shape adapted to place the distal electrode in contact with the ventricular septum and to place the proximal electrode in contact with the left ventricle spaced from the ventricular septum.

In some cases, the distal section may include, when unconstrained, a proximal spiral portion that defines a plane; and a distal spiral portion that curves out of the plane defined by the proximal spiral portion. In some cases, the distal electrode may be disposed on the distal spiral portion and the proximal electrode may be disposed on the proximal spiral portion. In some cases, the elongate shaft may include an uninsulated core wire that is electrically coupled with one of the distal electrode and the proximal electrode and a polymer-coated coil that is wrapped around the uninsulated core wire and that is electrically coupled with the other of the distal electrode and the proximal electrode. The proximal spiral portion may have a width that ranges from about 29 millimeters (about 1.14 inches) to about 50 millimeters (about 1.97 inches). The distal spiral portion may include a distal tip that extends out of the plane defined by the proximal spiral portion a distance ranging from about 5 millimeters (about 0.197 inches) to about 50 millimeters (about 1.97 inches).

A guidewire may be adapted for delivering a replacement heart valve implant while also being adapted to permit pacing the heart using the guidewire. The guidewire may include an elongate shaft including a distal section and a proximal section extending proximally from the distal section. The elongate shaft may have an outer diameter ranging from about 0.76 millimeters (about 0.030 inches) to about 1.02 millimeters (about 0.040 inches). The distal section may form a spiral portion when unconstrained. The spiral portion may include a proximal spiral portion that curves in a direction and defines a plane and a distal spiral portion that extends out of the plane defined by the proximal spiral. A proximal electrode is disposed within the proximal spiral portion and a distal electrode is disposed within the distal spiral portion.

In some cases, the proximal electrode and the distal electrode together may be adapted for pacing the heart. In some cases, the elongate shaft may include an uninsulated core wire that is electrically coupled with one of the distal electrode and the proximal electrode and a polymer-coated coil that is wrapped around the uninsulated core wire and that is electrically coupled with the other of the distal electrode and the proximal electrode. In some cases, the proximal spiral portion may have a width that ranges from about 29 millimeters (about 1.14 inches) to about 50 millimeters (about 1.97 inches). In some cases, the distal spiral portion may include a distal tip that extends out of the plane defined by the proximal spiral portion a distance ranging from about 5 millimeters (about 0.197 inches) to about 50 millimeters (about 1.97 inches).

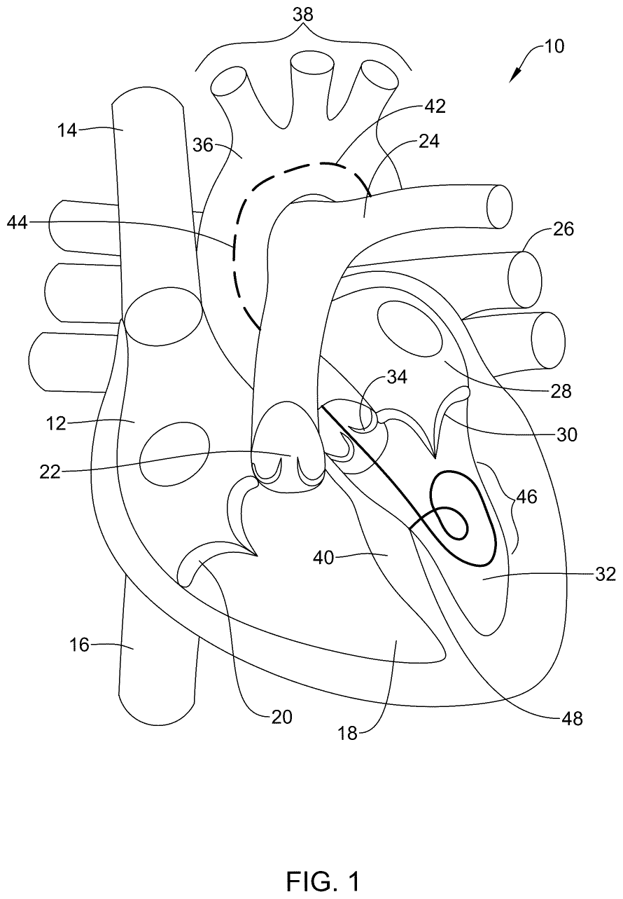

FIG. 1 is a schematic view of a human heart 10, illustrating some of the anatomy involved in accessing the interior of the heart 10 in general and in accessing the aortic valve in particular. The heart 10 includes a right atrium 12, which receives blood via the superior vena cava 14 and the inferior vena cava 16. Blood within the right atrium 12 passes to a right ventricle 18 via a tricuspid valve 20. Blood within the right ventricle 18 passes through a pulmonary valve 22 and into a pulmonary artery 24 in order to go to the lungs to be oxygenated. Oxygenated blood from the lungs returns via pulmonary veins 26 and enters a left atrium 28. A mitral valve 30 allows blood to flow from the left atrium 28 into a left ventricle 32. Blood exits the left ventricle 32, through an aortic valve 34, into an aorta 36 and from there is carried throughout the body. Other large arteries 38 (e.g., subclavian arteries, carotid arteries, brachiocephalic artery) extend from the aortic arch to important internal organs. A ventricular septum 40 separates the right ventricle 18 from the left ventricle 32.

A guidewire 42 passes down through the aorta 36 and passes through the aortic valve 34 to reach the left ventricle 32. As will be discussed and shown with respect to subsequent drawings, the guidewire 42 may be used to deliver and implant a replacement heart valve implant. For the purpose of this disclosure, the discussion below is directed toward the treatment of the native aortic valve 34 and will be so described in the interest of brevity. This, however, is not intended to be limiting as the skilled person will recognize that the following discussion may also apply to a mitral valve or another heart valve with no or minimal changes to the structure and/or scope of the disclosure. Similarly, the medical devices and/or procedures disclosed herein may have applications and uses in other portions of a patient's anatomy, such as but not limited to, arteries, veins, and/or other body lumens.

The guidewire 42 includes an elongate shaft 44. In some cases, the elongate shaft 44 may be relatively large in outer diameter in order to withstand the forces applied to the guidewire 42 when a replacement heart valve implant is advanced over the guidewire 42. As an example, the elongate shaft 44 may have an outer diameter that is in a range of about 0.76 millimeters (about 0.030 inches) to about 1.02 millimeters (about 0.040 inches). As another example, the elongate shaft 44 may have an outer diameter that is about 0.89 millimeters (about 0.035 inches). In some instances, as shown, the elongate shaft 44 may have a region 46 that forms a three dimensional shape 46 when the elongate shaft 44 is not otherwise constrained such as being within a guide catheter. The three dimensional shape 46 may permit the guidewire 42 to brace against a ventricular wall of the left ventricle 32 and to place a distal tip 48 of the guidewire 42 against the ventricular septum 40. Because the ventricular septum 40 includes pacing pathways, pacing at or near the ventricular septum 40 may be beneficial. In some cases, as will be discussed with respect to subsequent drawings, the guidewire 42 may include one or more electrodes that may be used in pacing the heart 10.

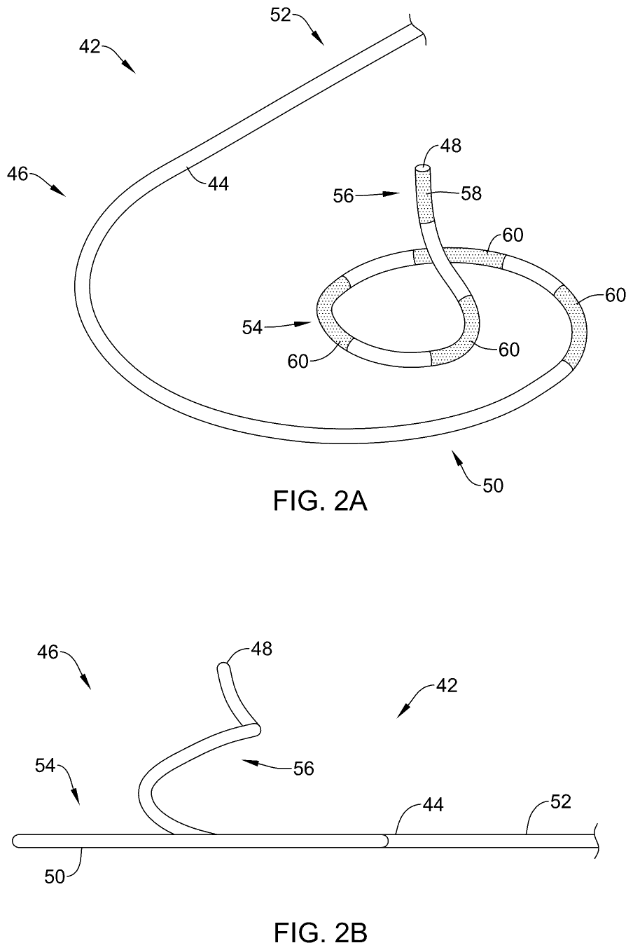

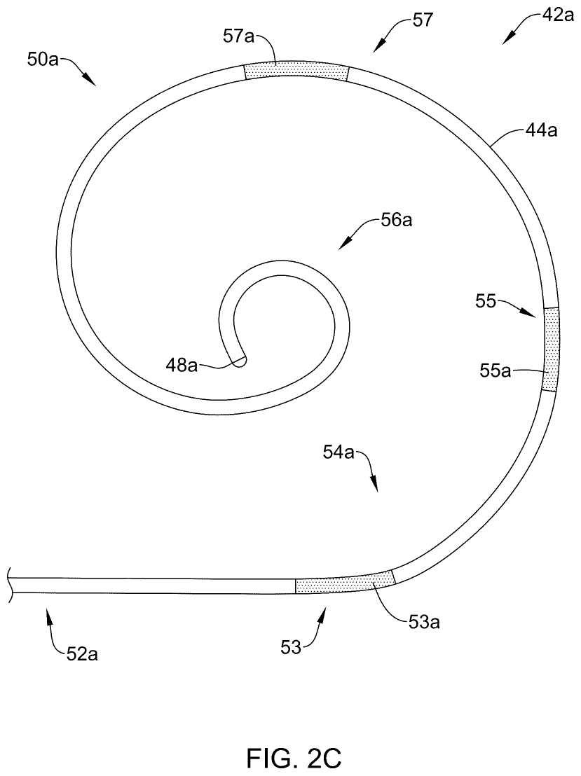

FIG. 2A is a perspective view of the guidewire 42 and FIG. 2B is a side view of the guidewire 42. The elongate shaft 44 includes a distal section 50 and a proximal section 52 that extends proximally from the distal section 50. In some cases, the three dimensional shape 46 may be limited to the distal section 50, i.e., the proximal section 52 may have a linear or substantially linear profile when not constrained by being inside a guide catheter, for example, while the distal section 50 or at least a substantial part of the distal section 50 may form the three dimensional shape 46. In some cases, the distal section 50 may include both a proximal spiral portion 54 and a distal spiral portion 56. The proximal spiral portion 54 may curve in a direction and may define a plane. As an example, the proximal spiral portion 54 may curve or spiral within the plane of the paper while the distal spiral portion 56 may curve or spiral out of the plane of the paper. Put another way, the proximal spiral portion 54 may be considered as curving or spiraling within an XY plane, while the distal spiral portion 56 may be considered as curving or spiraling in a Z direction relative to the XY plane. The distal spiral portion 56 could curve or spiral down through the plane of the paper, or up above the plane of the paper.

The proximal spiral portion 54 may have a variety of different sizes, depending on the dimensions of the heart 10 that the guidewire 42 is intended to be used in. As an example, a child's heart 10 may be smaller than an adult's heart 10. An adult male's heart 10 may be larger than an adult female's heart 10. In some cases, the proximal spiral portion 54, which may be considered as being within a single plane, may have an overall width (measured across the single plane) that ranges from about 29 millimeters (about 1.14 inches) to about 50 millimeters (about 1.97 inches). In an example, the proximal spiral portion 54 may about 29 millimeters (about 1.14 inches) by about 32 millimeters (about 1.26 inches). In another example, the proximal spiral portion 54 may be about 42 millimeters (about 1.65 inches) by about 42 millimeters (about 1.65 inches). In another example, the proximal spiral portion 54 may be about 49 millimeters (about 1.93 inches) by about 50 millimeters (about 1.97 inches). Similarly, the dimensions of the distal spiral portion 56 may vary as well. In some cases, the distal spiral portion 56 may have a height (measured in the Z direction above the XY plane) that ranges from about 5 millimeters (about 0.197 inches) to about 50 millimeters (about 1.97 inches). In some cases, the dimensions of the proximal spiral portion 54 and the dimensions of the distal spiral portions 56, including overall widths and heights, and degree of spiraling, may be similar to those of the Safari™ guidewire available commercially from Boston Scientific.

In some cases, the proximal spiral portion 54 and the distal spiral portion 56 may, in combination, exhibit a spiral that curves around itself at least 360 degrees, or at least 370 degrees, or at least 380 degrees, or at least 390 degrees, or at least 400 degrees, or more. Put another way, the proximal spiral portion 54 and the distal spiral portion 56 may, in combination, represent one or more revolutions around an arbitrary center point of the spiral. The proximal spiral portion 54 and the distal spiral portion 56 may, together, form two, three or more revolutions. In some cases, the degree of spiraling for the proximal spiral portion 54 and the distal spiral portion 56 in combination may be sufficient to allow at least part of the proximal spiral portion 54 to contact and brace against a ventricular wall of the left ventricle 32 while the distal spiral portion 56 extends sufficiently towards the ventricular septum 40 such that the distal tip 48, bearing an electrode, is able to make contact with the ventricular septum 40. In some instances, the proximal spiral portion 54 and the distal spiral portion 56 may, together, form a more closely wound spiral that includes additional revolutions. In some instances, the proximal spiral portion 54 and the distal spiral portion 56 may, together, for a more loosely wound spiral that includes fewer revolutions. This may vary depending on the anatomy of the patient, and can be part of providing multiple sizes of the guidewire 42.

In some instances, the distal section 50 may include one or more electrodes that may be used in pacing the heart 10. In some cases, the distal section 50 may include a single electrode that allows for unipolar pacing. During unipolar pacing, a return electrode would be placed somewhere on the patient's skin to create a return path. In some cases, the distal section 50 may include two or more electrodes that facilitate bipolar pacing. During bipolar pacing, a return path exists between one of the two or more electrodes and another of the two or more electrodes, as long as the one of the two or more electrodes are electrically isolated from the another of the two or more electrodes. As an example, the distal section 50 may include one or more anode electrodes and one or more cathode electrodes. As shown, the distal section 50 includes an anode electrode 58 and several cathode electrodes 60. The anode electrode 58 may have an overall length that ranges about 2 millimeters (about 0.079 inches) to about 50 millimeters (about 1.97 inches). The anode electrode 58 may be considered as being disposed within the distal spiral portion 56 and the cathode electrodes 60 may be considered as being disposed within the proximal spiral portion 54. In some cases, one or more of the cathode electrodes 60 may be disposed on the elongate shaft 44 proximal of the proximal spiral portion 54. There may be as many as ten cathode electrodes 60, although any other number such as one cathode electrode, two cathode electrodes, and so on through nine cathode electrodes are possible.

With brief reference to FIG. 1, the three dimensional shape 46, including the proximal spiral portion 54 and the distal spiral portion 56, may be positioned within the left ventricle 32 such that the proximal spiral portion 54 (bearing the cathode electrodes 60) may be positioned against the ventricular wall and the distal spiral portion 56 (bearing the anode electrode 58) may be disposed against the ventricular septum 40 and thus the anode electrode 58 may be well-positioned for pacing the heart 10.

FIG. 2C is a top plan view of a guidewire 42a, which may be considered as an example of the guidewire 42. FIG. 2C illustrates possible cathode electrode placement. Because this is a top view, the spiraling within the plane of the paper is visible, but the spiraling above the plane of the paper appears flat. In some cases, the elongate shaft 44a may be considered as having a transition 53 between a straight portion of the elongate shaft 44a and a curved section of the elongate shaft 44a. The elongate shaft 44a may be considered as having a transition 55 at the base of the curve where the elongate shaft 44a begins to spiral above the plane of the paper. The elongate shaft 44a may have a point 57 that is opposite or substantially opposite the transition 53, where substantially opposite may be defined as being within ten percent of opposite. In some cases, the guidewire 42a may include a cathode electrode 53a that is located at the transition 53. The guidewire 42a may include a cathode electrode 55a that is located at the transition 55. The guidewire 42a may include a cathode electrode 57a that is located at the point 57. The guidewire 42a may include additional cathode electrodes as well. In some cases, the cathode electrodes may be equally spaced apart. In some cases, the guidewire 42a may include an anode electrode that is disposed at or near the distal tip 48a.

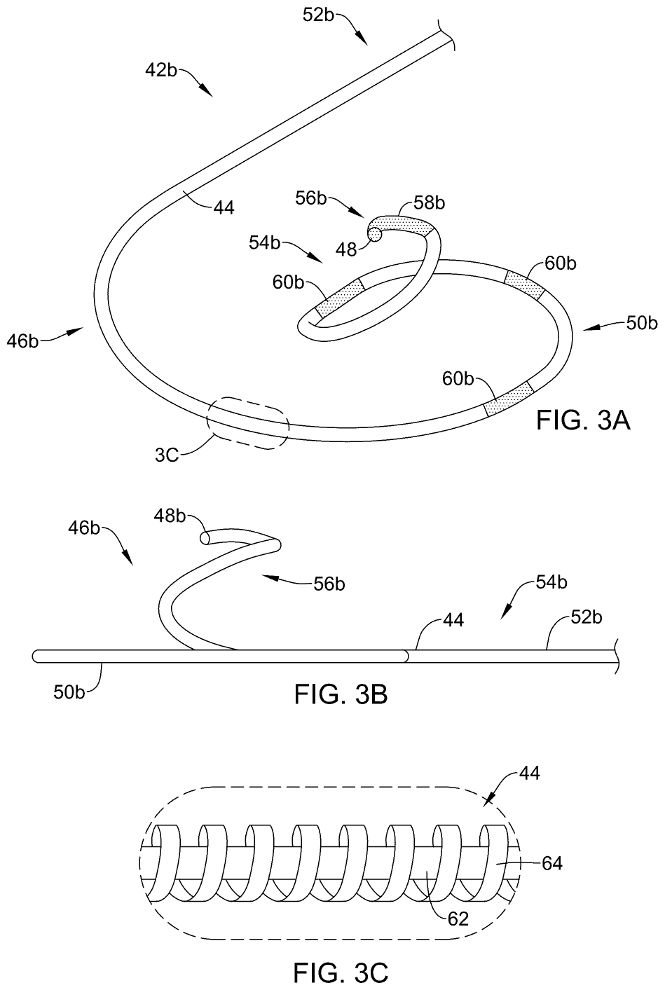

FIG. 3A is a perspective view of a guidewire 42b, which may be considered as an example of the guidewire 42 but with a different three dimensional shape 46b. While the three dimensional shape 46 and the three dimensional shape 46b provide two different examples of the three dimensional shape of the guidewire 42 (or 42b), it will be appreciated that other three dimensional shapes are contemplated, as long as the three dimensional shape is able to provide electrodes in appropriate positions to contact the desired portions of the cardiac anatomy. FIG. 3B is a side view of the guidewire 42b and FIG. 3C is an enlarged view of a portion of the shaft 44b. The elongate shaft 44b includes a distal section 50b and a proximal section 52b that extends proximally from the distal section 50b. In some cases, the three dimensional shape 46b may be limited to the distal section 50b, i.e., the proximal section 52b may have a linear or substantially linear profile when not constrained by being inside a guide catheter, for example, while the distal section 50b or at least a substantial part of the distal section 50b may form the three dimensional shape 46b. In some cases, the distal section 50b may include both a proximal spiral portion 54b and a distal spiral portion 56b. The proximal spiral portion 54b may curve in a direction and may define a plane. As an example, the proximal spiral portion 54b may curve or spiral within the plane of the paper while the distal spiral portion 56b may curve or spiral out of the plane of the paper. Put another way, the proximal spiral portion 54b may be considered as curving or spiraling within an XY plane, while the distal spiral portion 56b may be considered as curving or spiraling in a Z direction relative to the XY plane. The distal spiral portion 56b could curve or spiral down through the plane of the paper, or up above the plane of the paper.

In some instances, the distal section 50b may include one or more electrodes that may be used in pacing the heart 10. In some cases, the distal section 50b may include a single electrode that allows for unipolar pacing. During unipolar pacing, a return electrode would be placed somewhere on the patient's skin to create a return path. In some cases, the distal section 50b may include two or more electrodes that facilitate bipolar pacing. During bipolar pacing, a return path exists between one of the two or more electrodes and another of the two or more electrodes, as long as the one of the two or more electrodes are electrically isolated from the another of the two or more electrodes. As an example, the distal section 50b may include one or more anode electrodes and one or more cathode electrodes. As shown, the distal section 50b includes an anode electrode 58b and several cathode electrodes 60b. The anode electrode 58b may be considered as being disposed within the distal spiral portion 56b and the cathode electrodes 60b may be considered as being disposed within the proximal spiral portion 54b. With brief reference to FIG. 1, the three dimensional shape 46b, including the proximal spiral portion 54b and the distal spiral portion 56b, may be positioned within the left ventricle 32 such that the proximal spiral portion 54b (bearing the cathode electrodes 60b) may be positioned against the ventricular wall and the distal spiral portion 56b (bearing the anode electrode 58b) may be disposed against the ventricular septum 40 and thus the anode electrode 58b may be well-positioned for pacing the heart 10.

In some cases, the elongate shaft 44 may have a composite construction, which can facilitate making separate electrical connections to the anode electrode(s) 58 and 58b and to the cathode electrode(s) 60 and 60b. As an example, the elongate shaft 44 may include an inner core wire 62 and a polymer-coated coil 64 that is wrapped around the inner core wire 62. In some cases, the inner core wire 62 may be a bare wire and thus may not be insulated at all. In some cases, the polymer-coated coil 64 may be coated with an electrically insulative polymer. As an example, the polymer-coated coil 64 may be dip-coated with polytetrafluoroethylene (PTFE) which is electrically insulative and provides low friction characteristics.

The inner core wire 62 and the polymer-coated coil 64 may each be used as a electrically separate conductive path to the electrodes disposed within the distal section 50 and 50a. As an example, the inner core wire 62 may be electrically coupled with either the anode electrode(s) 58 and 58b or the cathode electrode(s) 60 and 60b and the polymer-coated coil 64 may be electrically coupled with the other of either the anode electrode(s) 58 and 58b or the cathode electrode(s) 60 and 60b.

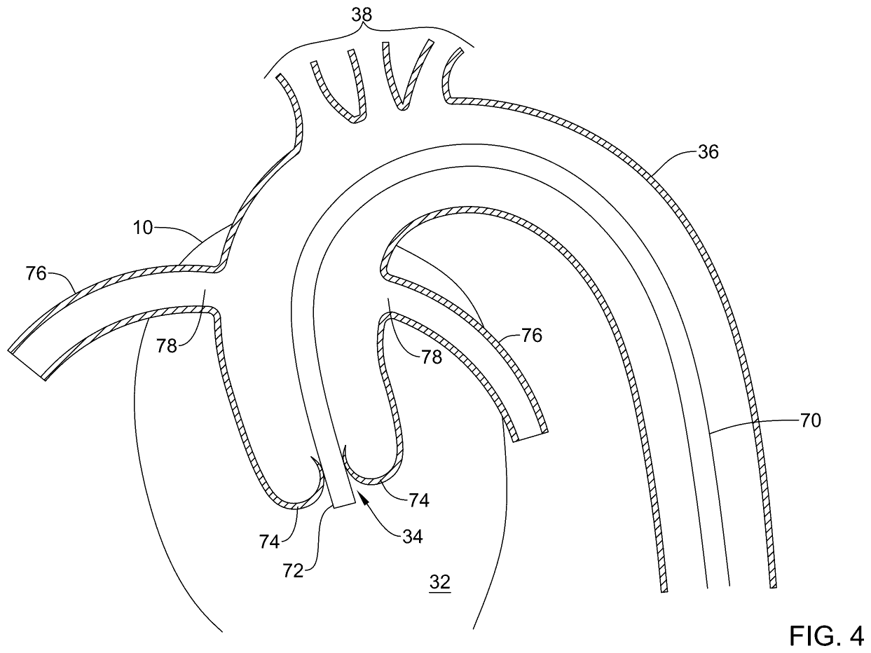

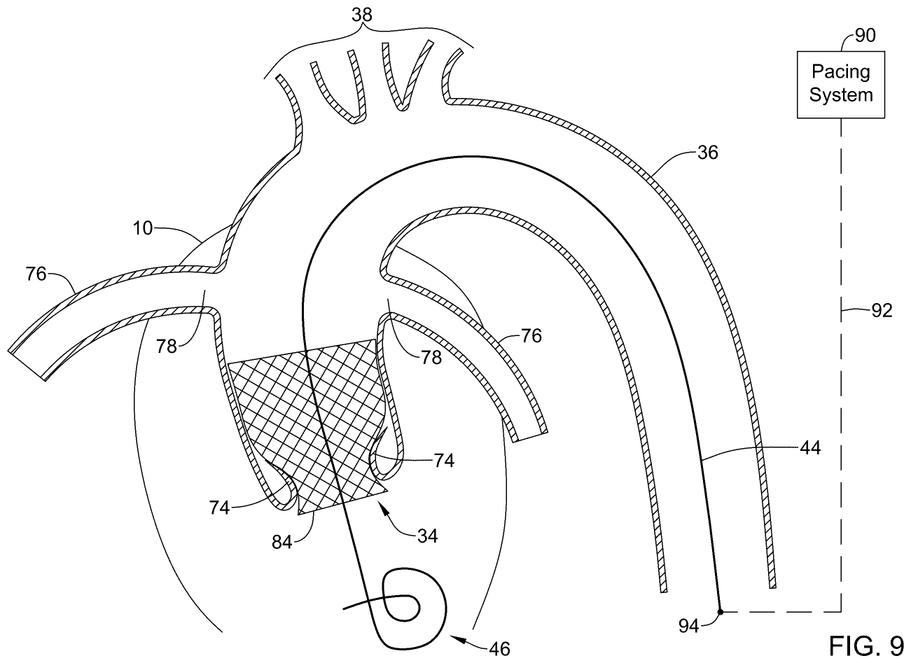

As noted, the guidewire 42 (or 42a or 42b) may be used for delivering and implanting a replacement cardiac valve within a patient. In some instances, the guidewire 42 (or 42a or 42b) may be adapted to also be able to be used for pacing the heart during the replacement valve implantation process. FIGS. 4 through 9 provide an illustrative but non-limiting example of using the guidewire 42 (or 42a or 42b) for delivery and implantation of a replacement cardiac valve while optionally using the guidewire 42 (or 42a or 42b) to deliver pacing pulses during the implantation process.

As seen in FIG. 4, a catheter 30 may be advanced through the patient's vasculature (e.g., the aorta 20, etc.) to and/or through the native heart valve (e.g., the aortic valve 34) into the left ventricle 32 of the patient's heart 10. A distal end 72 of the catheter 70 may extend through the valve leaflets 74 and may be disposed within the left ventricle 32 of the patient's heart 10. The heart 10 includes coronary arteries 76 and the ostia 78 of the coronary arteries 76. The catheter 70 may include one or more lumens extending through the catheter 70 to the distal end 72. At least one of the one or more lumens may be a guidewire lumen and/or a working lumen. In some embodiments, the patient's heart 10 may be accessed more directly and navigation through the patient's vasculature (e.g., the aorta 34) may not be necessary.

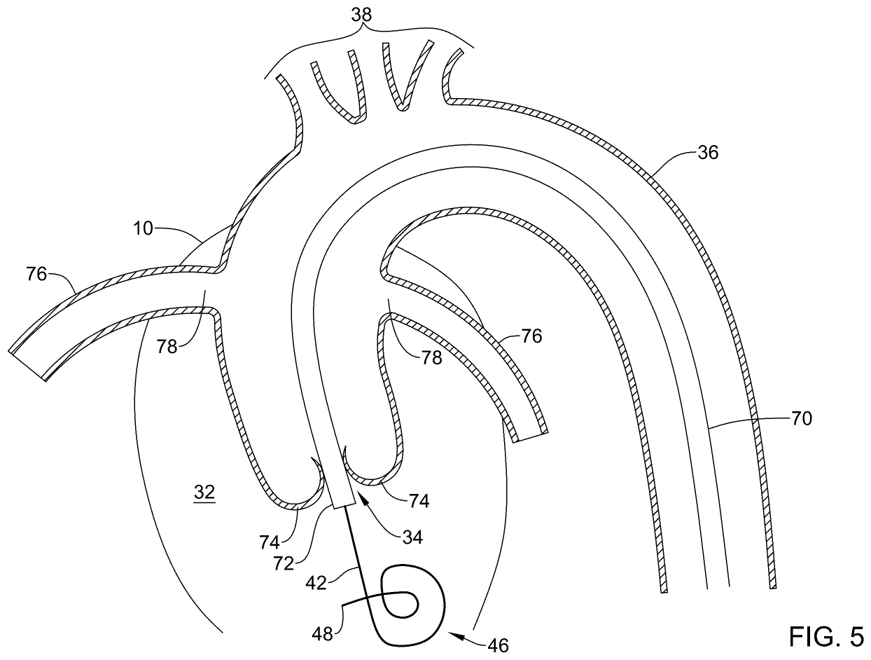

In some instances, the guidewire 42 (or 42a or 42b) may be advanced out the distal end 72 of the catheter 70 into the left ventricle 32 of the patient's heart 10, as seen in FIG. 5. The guidewire 42 (or 42a or 42b) may be configured as described herein. In some instances, the guidewire 42 (or 42a or 42b) may be contained and/or constrained in a straightened configuration within the one or more lumens of the catheter 70 as the catheter 70 is advancing to the native heart valve (e.g., the aortic valve 34) of the patient's heart 10. In some instances, the guidewire 42 (or 42a or 42b) may be advanced simultaneously with and/or within the catheter 70 through the patient's vasculature to the native heart valve (e.g., the aortic valve 34) of the patient's heart 10. In some instances, the guidewire 42 (or 42a or 42b) may be advanced through the catheter 70 after positioning the distal end 72 of the catheter 70 within the left ventricle 32 of the patient's heart 10. As the guidewire 42 (or 42a or 42b) exits the distal end 72 of the catheter 70, the guidewire 42 (or 42a or 42b) is no longer constrained, and the three dimensional shape 46 will reform.

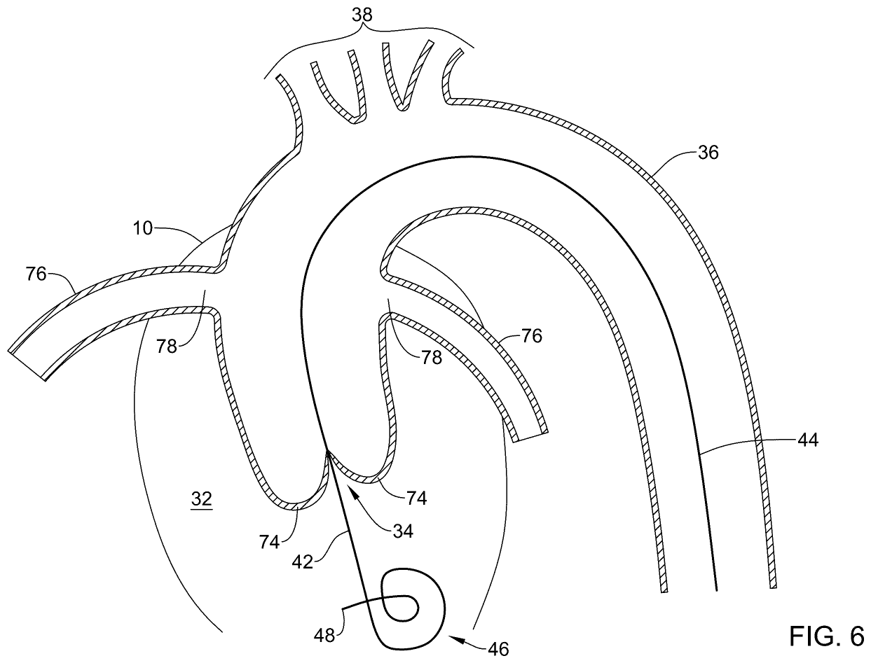

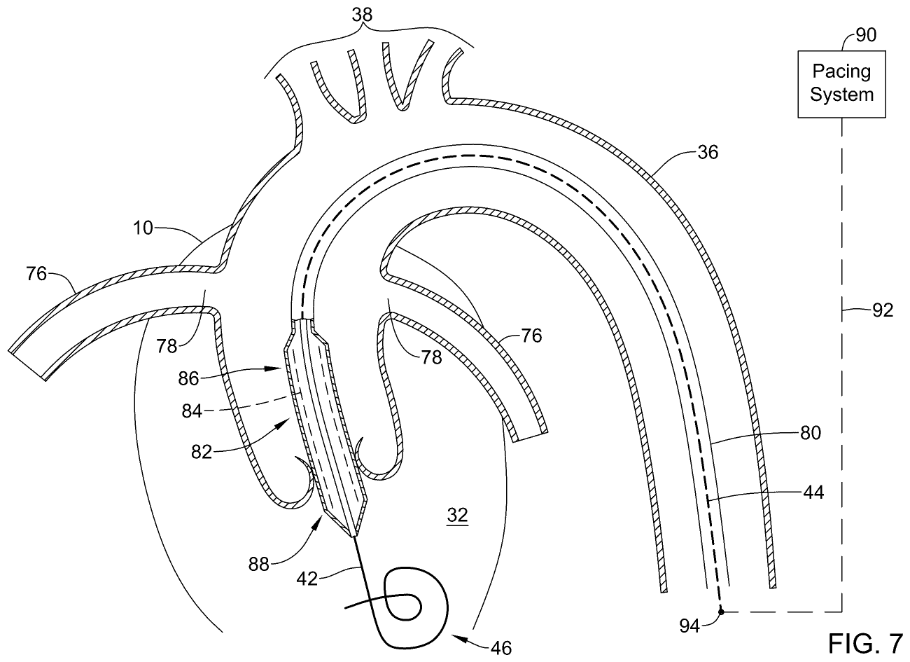

The catheter 70 may be removed while maintaining the three dimensional shape 46 of the guidewire 42 (or 42a or 42b) within the left ventricle 32 of the patient's heart 10, as shown in FIG. 6. In some instances, a deployment device 80 may be advanced over the guidewire 42 (or 42a or 42b) to the native heart valve (e.g., the aortic valve 34) of the patient's heart 10, as seen in FIG. 7. The deployment device 80 is shown in a partial cutaway view. In some instances, the deployment device 80 may include an outer sheath and an inner shaft axially translatable relative to the outer sheath. In some instances, the inner shaft of the deployment device 80 may include a central guidewire lumen extending therethrough to a distal end of the deployment device 80.

The deployment device 80 and/or the outer sheath may include a distal containment section 82 having a replacement heart valve implant 84 disposed therein in a constrained and/or collapsed configuration. The distal containment section 82 may include a proximal portion 86 and a distal portion 88. In some instances, the proximal portion 86 and the distal portion 88 of the distal containment section 82 may be configured to axially translate relative to each other to open the distal containment section and release the replacement heart valve implant 84.

In some instances, the proximal portion 86 of the distal containment section 82 may be fixedly attached to and/or integrally formed with the outer sheath. In some instances, the distal portion 88 of the distal containment section 82 may be fixedly attached to and/or integrally formed with the inner shaft. Therefore, relative axial translation between the outer sheath and the inner shaft may cause corresponding relative axial translation of the proximal portion 86 of the distal containment section 82 and the distal portion 88 of the distal containment section 82 to open the distal containment section 82 and release the replacement heart valve implant 84. The distal containment section 82 of the deployment device 80 may be positioned adjacent to and/or within the native heart valve (e.g., the aortic valve 34) prior to opening the distal containment section 82 of the deployment device 80. In at least some instances, the distal portion 88 of the distal containment section 82 may be disposed at least partially upstream of the native heart valve (e.g., the aortic valve 34) and/or at least partially within the left ventricle 32 of the patient's heart 10 prior to opening the distal containment section 82 of the deployment device 80.

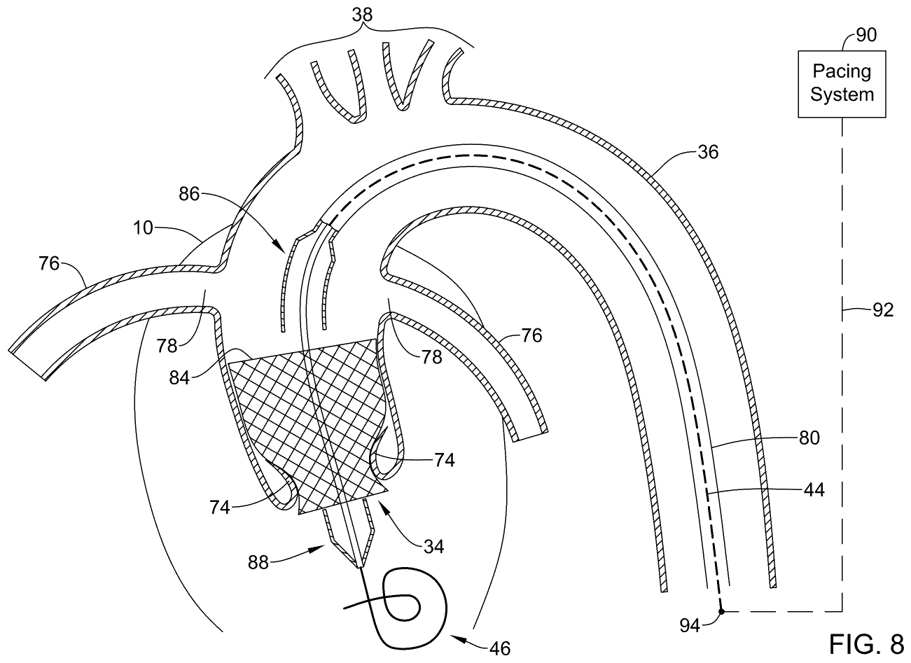

In some instances, the replacement heart valve implant 84 may be deployed within the native heart valve (e.g., the aortic valve 34) by opening the distal containment section 82 of the deployment device 80 while the distal containment section 82 is disposed within the native heart valve (e.g., the aortic valve 34), as seen in FIG. 8. The replacement heart valve implant 84 may include an expandable framework and a plurality of valve leaflets disposed within the expandable framework. In some instances, the expandable framework may be self-expanding. In some instances, the expandable framework may be mechanically and/or balloon expandable. Other configurations are also contemplated.

After opening the distal containment section, the distal portion 88 of the distal containment section 82 may be disposed upstream of the native heart valve (e.g., the aortic valve 34) and/or within the left ventricle 32 of the patient's heart 10 and the proximal portion 86 of the distal containment section 82 may be disposed downstream of the native heart valve (e.g., the aortic valve 34) and/or within the aorta 36 of the patient. Upon opening the distal containment section 82, the replacement heart valve implant 84 may shift toward and/or to an expanded deployed configuration. In some embodiments, the replacement heart valve implant 84 may be released from the distal containment section and/or the deployment device 80 prior to shifting to the expanded deployed configuration. In some instances, the replacement heart valve implant 84 may be released from the distal containment section and/or the deployment device 80 after shifting to the expanded deployed configuration.

In some instances, the distal containment section 82 may be closed prior to removing the deployment device 80 from the patient's heart 10 and/or vasculature. In some instances, the deployment device 80 may be disposed within and/or may be retracted into a delivery catheter (not shown) prior to removal from the patient's heart 10 and/or vasculature. Other configurations are also contemplated.

In some instances, the catheter 70, the deployment device 80, the distal containment section 82, and/or elements thereof may include at least one radiopaque marker for visualization during delivery and/or navigation through the patient's vasculature. The at least one radiopaque marker may permit accurate placement under fluoroscopy of the distal end 72 of the catheter 70 and/or the distal containment section 82 of the deployment device 80 with respect to the native heart valve (e.g., the aortic valve 34), the left ventricle 32, and/or the patient's heart 10.

In some instances, the physician or other professional implanting the replacement heart valve implant 84 may desire to use the guidewire 42 (or 42a or 42b) for delivering pacing pulses to the heart 10 during particular portions of the implantation process. In some instances, rapidly pacing the heart 10 during the implantation process may result in the heart 10 being less likely to displace the replacement heart valve implant 84 within the aortic valve 34. In some instances, as seen in FIGS. 7 through 9, a pacing system 90 may be utilized. The pacing system 90, which is shown schematically, may include a controller that determines when and how to pace, for example. The controller may also determine one or more pacing parameters for rapidly pacing the heart 10. An electrical cable 92 may extend from the pacing system 92, and may include a connector 94 that allows the electrical cable 92 to make an electrical connection with the guidewire 42 (or 42a or 42b). The connector 94 may take a variety of forms, depending on the construction of the proximal section 52 of the elongate shaft 44. The physician or other professional may utilize the pacing system 90 to pace during an appropriate step in implanting the replacement heart valve implant 84.

The materials that can be used for the various components of the guidewire, the catheter, the replacement heart valve implant, the deployment device, and/or the forming tool (and/or other elements disclosed herein) and the various components thereof disclosed herein may include those commonly associated with medical devices. For simplicity purposes, the following discussion makes reference to the medical device(s). However, this is not intended to limit the devices and methods described herein, as the discussion may be applied to other elements, members, components, or devices disclosed herein, such as, but not limited to, the elongate shaft, the outer sheath, the inner shaft, etc. and/or elements or components thereof.

In some embodiments, the medical device(s) and/or other elements disclosed herein may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®,UNS: N 10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N 10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DM TA) analysis over a large temperature range. For example, in some embodiments, there may be no martensite/austenite phase changes detectable by DSC and DM TA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the medical device(s) and/or other elements disclosed herein may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the medical device(s) and/or other elements disclosed herein. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device(s) and/or other elements disclosed herein to achieve the same result.

In some embodiments, a degree of Magnetic Resonance Imaging (MRI) compatibility is imparted into the medical device(s) and/or other elements disclosed herein. For example, the medical device(s) and/or components or portions thereof may be made of a material that does not substantially distort the image and create substantial artifacts (e.g., gaps in the image). Certain ferromagnetic materials, for example, may not be suitable because they may create artifacts in an MRI image. The medical device(s) or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the medical device(s) and/or other elements disclosed herein may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf A tochem), elastomeric polyamides, block polyamide/ethers, polyether block amide (PEBA, for example available under the trade name PEBA X®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PV dC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the medical device(s) and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePT FE), a polyolefinic material such as a polyethylene, a polypropylene, polyester, polyurethane, and/or blends or combinations thereof.

In some embodiments, the medical device(s) and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps, without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A guidewire for delivering a replacement heart valve implant, the guidewire comprising:
an elongate shaft including a distal section and a proximal section extending proximally from the distal section;
the distal section forming a spiral portion when unconstrained, the coiled portion including:
a proximal spiral portion that curves in a direction and defines a plane; and
a distal spiral portion that extends out of the plane defined by the proximal spiral; and
an electrode disposed within the distal section.

2. The guidewire of claim 1, wherein the electrode is adapted for pacing the heart.

3. The guidewire of claim 1, wherein the electrode comprises a first electrode that is disposed within the distal spiral portion.

4. The guidewire of claim 3, further comprising a second electrode disposed within the proximal spiral portion, the second electrode electrically isolated from the first electrode.

5. The guidewire of claim 4, wherein one of the first electrode and the second electrode comprises an anode electrode and the other of the first electrode and the second electrode comprise a cathode electrode.

6. The guidewire of claim 1, wherein the elongate shaft has an outer diameter that is in a range of about 0.76 millimeters (about 0.030 inches) to about 1.02 millimeters (about 0.040 inches).

7. The guidewire of claim 1, wherein the elongate shaft has an outer diameter that is about 0.89 millimeters (about 0.035 inches).

8. The guidewire of claim 1, wherein the elongate shaft comprises:
an uninsulated core wire; and
a polymer-coated coil wrapped around the uninsulated core wire.

9. The guidewire of claim 8, wherein one of the uninsulated core wire and the polymer-coated coil is electrically connected with the electrode.

10. A guidewire for delivering a replacement heart valve implant, the guidewire comprising:
an elongate shaft having an outer diameter ranging from about 0.76 millimeters (about 0.030 inches) to about 1.02 millimeters (about 0.040 inches), the elongate shaft including a distal section adapted for pacing;
a distal electrode disposed within the distal section;
a proximal electrode disposed within the distal section but spaced apart and electrically isolated from the first electrode;
the distal section having an unconstrained profile in which the distal section forms a three dimensional shape adapted to place the distal electrode in contact with the ventricular septum and place the proximal electrode in contact with the left ventricle spaced from the ventricular septum.

11. The guidewire of claim 10, wherein in the unconstrained profile, the distal section includes:
a proximal spiral portion that defines a plane; and
a distal spiral portion that curves out of the plane defined by the proximal spiral portion.

12. The guidewire of claim 11, wherein:
the distal electrode is disposed within the distal spiral portion; and
the proximal electrode is disposed within the proximal spiral portion.

13. The guidewire of claim 11, wherein the elongate shaft comprises:
an uninsulated core wire electrically coupled with one of the distal electrode and the proximal electrode; and
a polymer-coated coil wrapped around the uninsulated core wire and electrically coupled with the other of the distal electrode and the proximal electrode.

14. The guidewire of claim 11, wherein the proximal spiral portion has a width that ranges from about 29 millimeters (about 1.14 inches) to about 50 millimeters (about 1.97 inches).

15. The guidewire of claim 11, wherein the distal spiral portion includes a distal tip that extends out of the plane defined by the proximal spiral portion a distance ranging from about 5 millimeters (about 0.197 inches) to about 50 millimeters (about 1.97 inches).

16. A guidewire for delivering a replacement heart valve implant, the guidewire comprising:
an elongate shaft including a distal section and a proximal section extending proximally from the distal section, the elongate shaft having an outer diameter ranging from about 0.76 millimeters (about 0.030 inches) to about 1.02 millimeters (about 0.040 inches);
the distal section forming a spiral portion when unconstrained, the spiral portion including:
a proximal spiral portion that curves in a direction and defines a plane; and
a distal spiral portion that extends out of the plane defined by the proximal spiral;
a proximal electrode disposed within the proximal spiral portion; and
a distal electrode disposed within the distal spiral portion.

17. The guidewire of claim 16, wherein the proximal electrode and the distal electrode together are adapted for bipolar pacing of the heart.

18. The guidewire of claim 11, wherein the elongate shaft comprises:
an uninsulated core wire electrically coupled with one of the distal electrode and the proximal electrode; and
a polymer-coated coil wrapped around the uninsulated core wire and electrically coupled with the other of the distal electrode and the proximal electrode.

19. The guidewire of claim 16, wherein the proximal spiral portion has a width that ranges from about 29 millimeters (about 1.14 inches) to about 50 millimeters (about 1.97 inches).

20. The guidewire of claim 16, wherein the distal spiral portion includes a distal tip that extends out of the plane defined by the proximal spiral portion a distance ranging from about 5 millimeters (about 0.197 inches) to about 50 millimeters (about 1.97 inches).
